(19) United States
(12) Patent Application Publication
(10) Pub. No.: US 2026/0321416 A1
(43) Pub. Date: Oct. 15, 2026

(54) MEDICAL PUMP WITH CONTACTLESS DATA TRANSMISSION

(71) Applicant: B. Braun Melsungen AG, Melsungen (DE)

(72) Inventors: David Landmesser, Kassel (DE); Markus Discher, Knuellwald (DE)

(21) Appl. No.: 19/644,161

(22) Filed: Apr. 10, 2026

(30) Foreign Application Priority Data
Apr. 14, 2025 (EP) 25170456.5

(51) Int. Cl. G16H 40/67; A61M 5/172; G06K 7/10

(52) U.S. Cl. CPC G16H 40/67; A61M 5/172; G06K 7/10297; G06K 7/10366; A61M 2205/3584; A61M 2205/3592; A61M 2205/502; A61M 2205/52

(57) ABSTRACT

A medical pump includes a component for contactless data transmission of pump data to a mobile terminal device. The component, which can be an NFC chip or RFID chip, contains the pump data.

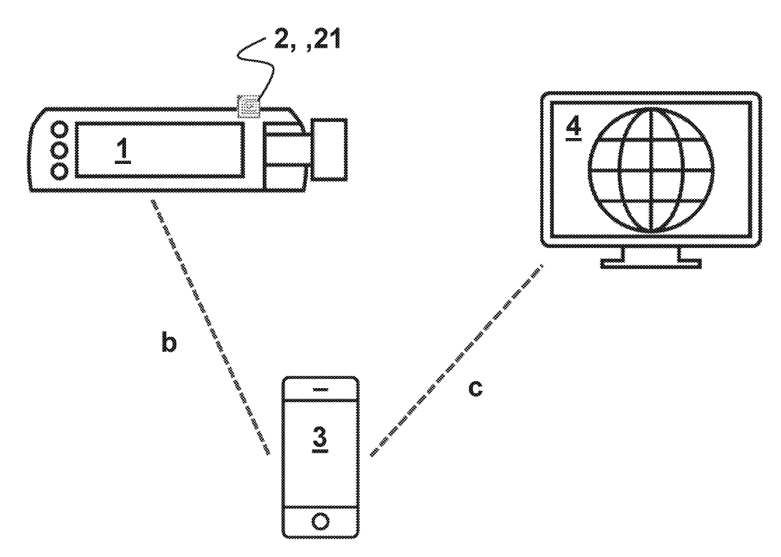

### CROSS-REFERENCE TO RELATED APPLICATION

This application claims priority under 35 U.S.C. § 119 to European Application No. 25170456.5, filed on Apr. 14, 2025, the content of which is incorporated by reference herein in its entirety.

### FIELD

The present disclosure relates to a medical pump, a method for wireless transmission of pump data, an app, and a system for wireless transmission of pump data.

### BACKGROUND

In the field of medical technology, medical pumps, such as infusion pumps or syringe pumps, are widely used and play a crucial role in the administration of drugs and other medical fluids to patients. The current state of the art includes various types of pumps that are capable of delivering precise dosages while ensuring a high level of reliability. These pumps are often equipped with various sensors and monitoring systems to increase their functionality and safety.

The problem is that, despite advances in technology, there are still significant challenges in using medical pumps. A key problem is the long time it takes to evaluate errors. When a pump fails or malfunctions, it can take a long time to identify and fix the exact cause.

The state of the art does include systems in which data from networked medical pumps can be read out via online applications. However, only trained medical technicians or employees of the pump manufacturers are permitted to read data via such online applications, and this can be done remotely, e.g., via VPN. This also poses the problem that, for security reasons, medical personnel without the appropriate education or training are not permitted to access the relevant software.

This makes it difficult to read data, especially for pumps that are not connected to a network. On the one hand, this is still the case in many hospitals worldwide. On the other hand, medical pumps are also used in non-networked applications, for example in transport (ambulances, helicopters), in doctors’ offices, or in day clinics.

In such scenarios, the error data must be read out and analyzed manually, which can be time-consuming and prone to errors. The pump is often removed and sent to appropriate service centers, especially the manufacturers, for initial analysis.

This leads to an increased workload for hospital staff. In addition, inadequate or delayed fault analysis can increase the risk of recurring problems, which also compromises the reliability of the pumps.

The hospital staff faces the challenge of quickly and efficiently solving technical problems and malfunctions that arise during operation. The need to manually read and analyze error data increases the workload, which is further exacerbated by the fact that manual analysis often requires specialized technical knowledge that is not always immediately available or that overwhelms existing staff.

In practice, there is additional effort involved, particularly in the context of recurring safety inspections (STK) and periodic maintenance and testing processes for medical pumps. This often involves identifying and documenting a large number of pumps, wherein device information such as serial number, date of manufacture, and pump type is typically read manually from the type plate and transmitted to medical

technology systems. In addition, a manual visual mark (e.g., a colored dot) can be used on the device to indicate the test status (e.g., “STK performed”).

Manually recording this device information is time-consuming, prone to errors, and ties up trained personnel. There is also a need to efficiently check and document test status or maintenance information directly on the device, even if the pump is not connected to a network and without necessarily having to remove or transport the device.

### SUMMARY

It is therefore one of the tasks underlying the present disclosure to overcome the disadvantages of medical pumps in the prior art. In particular, one of the underlying objectives of the present disclosure is to provide medical pumps and systems that feature improved error evaluation and data transmission.

Preferred embodiments can be found in the following description, particularly taking into account various embodiments as discussed and described herein.

The embodiments, features, and combinations of features described herein in connection with the present disclosure, and any combination of features mentioned and described in connection with the embodiments, are considered to be disclosed herein, or at least to be derivable by a person skilled in the art. In particular, each feature and each combination of features in the embodiments described herein may be in a different combination, at least because the skilled person will recognize that each individual combination of the features mentioned herein is suitable for contributing to the solution of the underlying problem.

Furthermore, each feature and each combination of features in the description below may be used separately, regardless of the subject matter category. For example, one or more embodiments according to the description below and/or the accompanying Figure may be provided in an arbitrary combination.

The tasks described above are solved according to the present disclosure by a medical pump according to the present disclosure. Such a medical pump comprises a component for contactless data transmission of pump data to a mobile terminal device, wherein the component comprises the pump data.

The term “the component comprises the pump data” is used here to express that the component for contactless data transmission has the ability to store and transmit relevant pump data. This pump data may include information such as error data, error analysis data, device status data, and device type data. This data is stored and transmitted securely and efficiently to ensure reliable communication with a mobile terminal device. It is preferable that the data is stored at least temporarily in the component for contactless data transmission. Within the scope of the present disclosure, it is possible that the data is originally generated and/or stored elsewhere, for example in a control unit, before being forwarded to the component for contactless transmission.

According to a preferred embodiment, the pump data additionally or alternatively includes identification data of the medical pump, in particular a serial number, a date of manufacture, and/or a pump type, preferably in a form that allows the device to be uniquely identified. This means that contactless data transmission can be used not only for troubleshooting, but also for service and testing processes that require fast, error-free identification of the device.

In another preferred embodiment, the pump data additionally or alternatively includes status information on a service or inspection status of the pump, for example information on whether a safety inspection (STK) or other inspection/maintenance has already been carried out and/or whether such an inspection is due. This allows users to read and document the pump’s test status contact-free using a mobile terminal device, without having to manually record type plate data or apply or evaluate visual markings on the device.

In another preferred embodiment, the component for contactless data transmission is designed to enable not only the reading of pump data but also the contactless reception of data from the mobile terminal device, in particular within the framework of bidirectional contactless communication. This allows data entries generated or provided by the mobile terminal device to be stored at least partially on the component. Such data entries may include, in particular, the service and/or inspection status of the medical pump, for example, information that an inspection, maintenance, or safety check has been performed and/or when such a measure was performed. This allows an updated status to be assigned directly to the medical pump and read out later without contact, which can support service, maintenance, and/or testing processes.

The tasks described above are also solved by an app according to the present disclosure. Such an app is designed to display to the user, preferably on a mobile terminal device, the pump data transmitted from a contactless data transmission component of a medical pump to the mobile terminal device. If necessary, the app can also analyze or evaluate some or all of the pump data. The present disclosure also envisages that, in addition or alternatively, the app is configured to transmit the pump data transmitted from the medical pump to the mobile terminal device by the contactless data transmission component to a service facility. This increases user-friendliness and significantly reduces the likelihood of errors caused by user mistakes. The automatic transmission of data also enables the service facility to analyze and process the data in a timely manner.

In a particularly preferred embodiment of the present disclosure, the app is configured to filter and/or sort the retrieved data on the mobile terminal device.

This feature significantly improves the efficiency and accuracy of data analysis. Users can extract specific relevant information from a large amount of data, which can simplify the diagnosis of the medical pump. In addition, the filter and sort function enables quick identification of errors or anomalies, which shortens the response time in the event of problems.

In another preferred embodiment of the present disclosure, the app is configured to send a notification to the user once the data has been successfully transmitted.

This feature enhances user-friendliness and security by immediately informing the user that the data transmission has been successfully completed. This ensures that the user is informed about the status of the data transmission and can react if necessary, especially in the event of an incomplete data transmission.

In another preferred embodiment of the present disclosure, the app is configured to add and transmit a user-defined comment to the transmitted data. This feature allows the user to customize and additionally contextualize data. The user can add specific notes or comments to the transmitted data that are relevant for further analysis or documentation. This contributes to improving communication and cooperation between the user and the service provider, as additional information is provided that facilitates the interpretation and processing of the data.

In another preferred embodiment of the present disclosure, the app has an interface for integration with other hospital information systems. This allows the transmitted pump data to be integrated directly into the central information systems and processed there. This leads to improved data availability and consistency, as all relevant information is consolidated in one place.

In a preferred embodiment, the app is configured to extract identification data (e.g., serial number, pump type) and/or service or test status information (e.g., STK status) from the contact-free read pump data and display it in a test or service log. The app can be set up to automatically transmit this data to medical technology documentation and/or to another IT system, such as a PC workstation or hospital information system, in order to reduce documentation effort and avoid input errors.

Furthermore, the app is configured to display a note or label indicating whether a pump has already been recorded as part of a testing process and/or whether a test status is available. This allows users to receive immediate feedback when recording multiple pumps without having to rely on manual markings or subsequent comparisons.

In particular, the app may, according to the present disclosure, include a communication option between the service facility and the app user, e.g., screen mirroring and/or a chat option may allow employees of the service facility to directly access the app and/or the pump data or to share information with the app user, preferably in real time.

The tasks described above are also solved according to the present disclosure by a system for wireless transmission of pump data according to the present disclosure. Such a system according to the present disclosure comprises a medical pump according to the present disclosure, which comprises a component for contactless data transmission of pump data to a mobile terminal device, wherein the component comprises the pump data. The system also includes a mobile terminal device that serves as a reader for the pump data for contactless data transmission from the transmission component to the mobile terminal device, and an app according to the present disclosure. The system also includes a service facility that receives pump data from the mobile terminal device and, preferably, analyzes it. It should be understood that both the app and the service facility can be set up to perform analyses and/or partial analyses simultaneously or independently of each other.

The tasks described above are also solved according to the present disclosure by a method for wireless transmission of pump data according to the present disclosure. Such a method according to the present disclosure comprises:
step a) of providing a medical pump, preferably a medical pump according to the present disclosure, with a component for contactless data transmission,
step b) of transmitting pump data from the medical pump to a mobile terminal device using the contactless data transmission component.

Step b) of transmitting pump data from the medical pump to a mobile terminal device using the contactless data transmission component may preferably include a step of checking or validating the data transmission. In particular, this ensures error-free data transmission or alerts users to faulty data transmission. For example, the error-free transmission of data or data packets can be validated using a checksum.

It is preferable that the pump data be structured in a data packet suitable for transmission or storage. Structuring data into appropriate packets can improve data integrity by minimizing the risk of data loss or corruption during transmission. In addition, the transmission speed increases because the data is available in a standardized format, which enables faster and more efficient transmission. Structured data packages also facilitate the processing and analysis of data by downstream systems or applications. In case of problems, structured data packets can be checked and analyzed more easily to identify sources of error. In particular, the provision and transmission of data packets allows components for data transmission to be used within the scope of the present disclosure which may not be suitable for the transmission of larger amounts of data.

It is further preferable that the data packet comprises a time segment around an event and/or specific measured values relating to components such as the drive or the electronics.

This enables targeted and precise analysis of the relevant data, as specific events or measured values can be isolated and examined. Focusing on specific time periods or measured values facilitates troubleshooting and increases diagnostic accuracy. In addition, resources can be used more efficiently through the targeted selection of data segments, as only relevant data is transmitted and stored.

In a preferred embodiment, the method additionally comprises a step of reading out protocol or log data from the medical pump (in particular a log file) by the mobile terminal device by means of the contactless data transmission component, wherein the reading out preferably takes place on site at the place of use of the pump. The log data read out can then be displayed in the app on the mobile terminal device and/or subjected to an initial analysis to support an initial classification of a fault or event.

In addition or alternatively, the app can process the read log data in an analysis mode, in particular by filtering, sorting, and/or creating a report from the read data. This enables the user to perform an initial technical assessment without necessarily having to transport the pump or access a stationary evaluation environment, although further analysis can still be carried out by a service facility.

Preferably, the medical pump and/or the component are designed so that the pump data can be read without power, especially in the event of a power failure.

The term “contactless” as used herein preferably refers to the transmission of data or energy without a physical connection between the devices involved. In this context, “contactless” means “directly contactless,” so that the transmission takes place immediately and without any physical connection between the component and the mobile terminal device. This excludes indirect contactless transmissions in which data or energy is transmitted via intermediate stations or additional devices. An example of direct contactless transmission is the use of near-field communication (“NFC”) or radio frequency identification (“RFID”) technology, in which data is exchanged directly between the pump and the mobile terminal device.

The term “medical pump,” as used herein, preferably refers to a device used for the controlled administration of fluids, such as medications or nutrient solutions, to patients. There are various types of medical pumps, each designed for specific applications and requirements. Common medical pumps include, for example, infusion pumps, which are used for the continuous or intermittent administration of fluids over a longer period of time; insulin pumps, which are specifically designed for the continuous delivery of insulin to diabetes patients; pain pumps, also known as patient-controlled analgesia (“PCA”) pumps, which allow patients to administer pain medication independently; feeding pumps, which are used to administer nutritional solutions to patients who are unable to consume sufficient food orally; Syringe pumps, which enable precise dosing of medications; and Heart pumps, which are used to support heart function.

Medical pumps are often equipped with various sensors and monitoring systems to ensure their functionality and safety. The pumps generate various data that may be necessary for analyzing the pump, in particular device functionalities, performance, or errors. According to the present disclosure, such pump data includes, among other things, error data, error analysis data, device status data, device type data, performance data, time stamps, input logs, alarms, and information about a disposable item used.

A timestamp can be particularly advantageous in determining and/or classifying the time of an event or other pump data entry. In other words, the timestamp can be used to determine exactly when something happened. An input log, as used herein, is preferably understood to mean that the input is logged by a user. For example, the input log records which keys a user has pressed. Alarms can be recorded in the pump data both by time and by type. In particular, alarms can be recorded in different alarm types, e.g., as operational alarms, device alarms, pre-alarms, or caution alarms.

The present disclosure provides a medical pump that includes a component for contactless data transmission of such pump data to a mobile terminal device. This offers numerous advantages. For example, real-time data transmission allows pump data to be sent immediately to the mobile terminal device, enabling immediate monitoring and analysis. This is particularly important in order to be able to respond quickly to any malfunctions or deviations. Contactless data transmission also increases efficiency and usability for hospital staff, as manual data reading and transmission is no longer necessary. The pump can remain on site while the pump data is read out and analyzed, e.g., for fault analysis. This saves time and significantly reduces the workload of hospital staff.

Another advantage is increased mobility and flexibility. Since the data is transmitted to a mobile terminal device, it can be viewed and analyzed from anywhere. The immediate availability of pump data allows errors to be identified and corrected more quickly, reducing pump downtime and improving continuity of patient care. In particular, the present disclosure allows the data to be read out in a particularly simple manner, and thus also by untrained personnel. Furthermore, reading data does not depend on whether or not there is a connection to a network, in particular the Internet. The data can be read manually, even if the medical pump is not online.

In connection with the method according to the present disclosure, it should also be noted that the steps indicated do not necessarily have to be carried out in the order specified. The steps listed can be performed in any other suitable order or even simultaneously.

However, the order specified above may be advantageous for certain embodiments of the method according to the present disclosure.

It will also be recognized by a person skilled in the art that a feature, embodiment, effect, or advantage as described here in connection with the inventive device and/or the inventive app and/or the inventive system may also be a feature, embodiment, effect, or advantage of the inventive method, and vice versa.

The term “contactless data transmission component,” as used herein, preferably refers to a component or unit that is capable of transmitting data without a physical connection. This component can include various technologies, such as NFC or RFID, which enable data to be sent and received wirelessly to a mobile terminal device.

Such components are particularly useful in medical pumps, as they enable fast and efficient transmission of pump data. This reduces the need for manual intervention and minimizes the risk of transmission errors. In addition, these components can be equipped with security features to prevent unauthorized access to data, which increases data security. Contactless data transmission thus offers improved user-friendliness and increases efficiency and safety in the medical environment.

The term “mobile terminal device,” as used herein, preferably refers to a portable electronic device capable of wireless communication and data processing. Such devices typically include smartphones, tablets, laptops, and similar portable computers equipped with appropriate interfaces for receiving and processing data from other devices, such as medical pumps.

These mobile terminal devices are capable of receiving data via various wireless technologies such as NFC, RFID, Bluetooth, or Wi-Fi. They offer a user-friendly interface for displaying and analyzing the received data and enable hospital staff to access important information quickly and efficiently. The use of mobile terminal devices increases the flexibility and mobility of hospital staff, as they can view and manage data from anywhere.

In a preferred embodiment of the present disclosure, the component for contactless data transmission comprises an NFC chip and/or an RFID chip that is set up to read the pump data using the mobile terminal device.

This is particularly advantageous because the use of an NFC chip and/or an RFID chip in the component for contactless data transmission enables fast and reliable transmission of the pump data. These technologies are known for their high transmission speed and reliability, which means that data can be transmitted to mobile terminal devices in near real time. This allows hospital staff to immediately access important information and respond accordingly or forward it directly to the service facility.

Within the scope of the present disclosure, the medical pump preferably comprises a microprocessor, microcontroller, and/or other computing unit for generating the pump data and/or, if necessary, for transmitting the pump data to the component.

In one embodiment of the present disclosure, the medical pump is configured to collect pump data and store it on the component. The data can be collected and/or stored on the component regularly, i.e., at regular intervals or when the data is generated.

In another preferred embodiment of the present disclosure, the medical pump is designed to record pump data on a user-based and/or event-based basis.

This is particularly advantageous because the medical pump enables more precise and individualized monitoring and control through user-based and/or event-based recording of pump data. In the context of the present disclosure, user-based data collection means, as used herein, that the pump collects data generated by the user. This may include, for example, adjusting the pump output to the specific needs and behavior of the respective user, resulting in optimized and personalized treatment.

Event-based data acquisition, on the other hand, means in the context of the present disclosure, as preferably used herein, that the pump responds to certain events or conditions, in particular errors, and acquires corresponding data. This may be the case, for example, if an error occurs or the operating status of the pump changes. By recording such events, potential problems can be identified and resolved at an early stage before they lead to major disruptions. The combination of both approaches offers a comprehensive and flexible solution for data acquisition that increases both the efficiency and safety of the pump. Users, particularly hospital staff, emergency services, or others, can access detailed and relevant data that helps them make informed decisions and improve patient care.

In another preferred embodiment of the present disclosure, the pump is designed to store pump data on the contactless data transmission component on a user-based and/or event-based basis. This means that the pump is capable of collecting and storing data based on user actions or specific events. User-based data can be generated, for example, by user input or interactions, while event-based data is collected by specific events or conditions of the pump. This data is then stored on the contactless data transmission component to enable efficient and secure transmission to a mobile terminal device. In other words, the present disclosure envisages that a user can specifically trigger the storage of data on the contactless data transmission component on a user-based basis. At the same time or alternatively, certain, possibly predetermined events—event-based—such as an alarm, a power failure, or similar, can trigger the storage of data on the component for contactless data transmission.

This is particularly advantageous because storing pump data on the contactless data transmission component enables efficient and flexible data management based on both user and event data. In particular, a log file containing pump data can be created.

Storing the data directly on the contactless data transmission component offers the additional advantage that the data is available anytime, anywhere, without the need for an internet connection. This increases the flexibility and mobility of hospital staff, as they can easily read and analyze the data using a mobile terminal device.

In another preferred embodiment of the present disclosure, the transmission of pump data from the component for contactless data transmission to a mobile terminal device is independent of the pump’s Internet connection.

In another preferred embodiment of the present disclosure, the component for contactless data transmission is protected by at least one security feature against unauthorized reading of the pump data by means of the mobile terminal device.

The term “security feature,” as used herein, preferably refers to a measure or method that serves to prevent unauthorized access to data or systems. Such security features may take various forms, including, but not limited to, passwords, targeted authorizations, and activation codes. A password may be required to enable access to the data, while targeted authorizations ensure that only authorized users have access to the pump data. An activation code can serve as an additional security level that must be entered before the data can be read. These security features help to ensure the integrity and confidentiality of pump data by preventing unauthorized access and ensuring that only authorized persons can view and use the data.

A security feature may preferably be provided on the medical pump according to the present disclosure and/or in the app according to the present disclosure.

The term “interference,” as used herein, preferably refers to any type of unwanted influence or disruption that negatively affects the functionality or performance of a device or system. This may include electromagnetic interference, mechanical vibrations, temperature changes, or other external influences that could impair the proper functioning of the contactless data transmission component.

In another preferred embodiment of the present disclosure, the component for contactless data transmission is arranged in an interference-shielded area of the medical pump.

This means that the component is protected by special measures or materials that prevent external interference from affecting data transmission.

This shielding is particularly advantageous as it increases the reliability and accuracy of data transmission. Protection against interference ensures that pump data can be transmitted correctly and without interruption to the mobile terminal device. This contributes to the overall reliability of the medical pump and ensures that hospital staff have access to accurate and reliable data at all times.

In a further preferred embodiment of the present disclosure, in step c), the pump data read out in step b) is transmitted from the mobile terminal device to a service facility. The term “service facility,” as used herein, preferably refers to a facility or system used for processing, analyzing, and monitoring data. A service facility can be a server, a computer, a central data management system, a cloud platform, or similar, but also another medical device that uses the data for further analysis or monitoring.

This facility serves as a central location where data can be collected, stored, and analyzed. Transmitting data to a service facility enables comprehensive and efficient data processing, which contributes to improving patient care and optimizing pump performance.

In another preferred embodiment of the present disclosure, the transmission of the pump data to the service facility is encrypted in step c). The term “encrypted,” as used herein, preferably refers to a method of data transmission in which the data is converted into a coded form to protect it from unauthorized access. This encryption ensures that only authorized recipients who have the appropriate decryption key can read and process the data.

In another preferred embodiment of the present disclosure, the pump data read out in step b) is displayed in an app on the mobile terminal device. This is particularly advantageous because displaying the pump data read out in step b) in an app on the mobile terminal device enables immediate and user-friendly visualization of the data. By using an app, the data can be displayed in real time, allowing fast and efficient monitoring of pump performance and device status. The app offers an intuitive user interface that makes it easier for hospital staff to quickly capture and interpret relevant information. This reduces the need for extensive training and allows staff to focus on patient care. In addition, the app can be used to set up user-defined alarms and notifications that immediately alert staff to critical conditions or errors in the medical pump.

Another advantage is the mobility and flexibility achieved by using a mobile terminal device and an app. Clinic staff can view and manage pump data from anywhere, increasing efficiency and responsiveness. In addition, the data can be stored in the app and used for later analysis or reports, which supports long-term monitoring and optimization of pump performance.

In another preferred embodiment of the present disclosure, the pump data transmitted in step c) is evaluated, preferably in the service facility.

The term “evaluate,” as used herein, preferably refers to the process of analyzing and interpreting data in order to obtain relevant information and insights. This means that once the pump data has been transmitted to the service facility, it is systematically analyzed to identify faults, monitor pump performance, or detect trends and patterns, for example. The evaluation can include various methods and techniques, such as statistical analyses, machine learning, or other data analysis methods, in order to comprehensively interpret the data and make informed decisions.

The evaluation of data at the service facility provides detailed insights into the pump’s functionality and condition and enables faults to be identified, which helps to improve the pump’s reliability and efficiency and can provide information on how to rectify the fault.

The following describes further embodiments of the present disclosure, wherein these embodiments may be provided individually or in any combination with each other and/or with the features described above.

In one embodiment, the pump data comprises identification data of the medical pump, in particular a serial number and/or a pump type and/or a date of manufacture, wherein this identification data is designed to be read contact-free by means of the mobile terminal device in order to enable unambiguous identification of the medical pump.

In one embodiment, the pump data includes a service and/or inspection status of the medical pump, in particular a status of a safety inspection and/or maintenance, wherein the service and/or inspection status can be read contact-free by means of the mobile terminal device in order to enable a determination as to whether an inspection has been carried out, is being carried out, and/or is due.

In one embodiment, the app is designed to collect, display, and/or store identification data and/or service and/or test status data from the medical pump in a contactless manner and/or to transmit it to a service facility and/or an external data processing system, in particular to support a service, maintenance, and/or testing process.

In one embodiment, the mobile terminal device is configured to use the contactless data transmission component to read log data from the medical pump without contact and to display this log data in the app and/or analyze it at least in part, in particular to enable an initial analysis of an operating state and/or a fault in the medical pump.

In one embodiment, the method comprises a step of contact-free reading identification data from a plurality of medical pumps using the mobile terminal device, wherein the read identification data is automatically stored and/or transmitted to a service facility and/or an external data processing system to support documentation and/or a service, maintenance, and/or inspection process.

In one embodiment, the component for contactless data transmission is designed to transmit pump data contact-free while the medical pump is in its intended location, in particular without the medical pump having to be removed or physically connected to an external reading device.

In one embodiment, the component for contactless data transmission is designed not only to transmit pump data to the mobile terminal device without contact, but also to receive data from the mobile terminal device without contact and to store at least some of it on the component, particularly in the context of bidirectional contactless communication.

In one embodiment, the mobile terminal device and/or the app is configured to transmit a service and/or test status of the medical pump, in particular information about a completed or pending safety inspection and/or maintenance, to the component for contactless data transmission in a contactless manner, so that the service and/or test status can be stored on the component and read out contact-free at a later point in time.

### BRIEF DESCRIPTION OF THE DRAWING

The present disclosure will be explained in more detail below with reference to the Figure, from which further features, embodiments, and advantages can be derived.

The Figure shows a schematic representation of a system according to a first embodiment of the present disclosure.

### DETAILED DESCRIPTION

The Figure shows a schematic representation of a system for wireless transmission of pump data according to a first embodiment of the present disclosure. The system comprises a medical pump 1 according to the present disclosure, which comprises a component 2 for contactless data transmission of pump data to a mobile terminal device 3. This component 2 for contactless data transmission can be, for example, an NFC chip 21 and/or an RFID chip 21, or comprise an NFC chip 21 and/or an RFID chip 21. The Chip 21 is set up to read the pump data using the mobile terminal device 3, in this case a smartphone 3. In the case of an NFC chip 21, bidirectional communication between chip 21 and terminal device 3 can be used to both send and receive data. If the chip 21 is an RFID chip, it is usually unidirectional, meaning that data can only flow from the RFID chip 21 to the RFID reader, in this case the smartphone 3 or a reader in the smartphone 3. The Chip 21 can be advantageously located in an area of the medical pump 1 that is shielded from interference.

Component 2, or chip 21, contains the pump data for pump 1. Pump 1 can record the pump data, in particular on a user-based and/or event-based basis.

The system also includes mobile terminal device 3, which serves as a reader for the pump data for contactless data transmission from chip 2 to mobile terminal device 3. An app based on the present disclosure is installed on the terminal device 3, e.g., a smartphone. Via this app, the service facility 4 can receive pump data from the smartphone 3 and then analyze it. Different pump data can be used and analyzed, in particular error data, error analysis data, device status data, or even device type data for pump 1.

The app on smartphone 3 is designed to display the pump data transmitted from chip 2 of medical pump 1 to smartphone 3, either additionally or alternatively, and to analyze it.

The app can also transmit the pump data transmitted from chip 2 of medical pump 1 to smartphone 3 to service facility 4. For this purpose, an Internet connection between the service facility 4 and the mobile terminal device 3, i.e., in this case the smartphone, is preferably used.

In one embodiment, the system shown in the Figure can also be used to support service and inspection processes, in particular recurring safety inspections. In this case, mobile terminal device 3 uses component 2 to read not only error or status data, but also, preferably, identification data for pump 1 (e.g., serial number, pump type) and/or a service or test status. As a result, a user can efficiently capture a plurality of pumps and document the captured data in the app and/or transmit it to service facility 4 or another IT system.

The app can preferably provide feedback on whether a test status is already available for the respective pump 1 or whether the pump has already been recorded as part of the process. This allows immediate plausibility checking of the data entry without the need to manually transmit type plate information or add additional visual markings to the device.

According to the method of the present disclosure, the medical pump 1 provided in step a) will first store the pump data on the chip 2 and then, in step b), which is shown in the Figure as a schematic dashed line b, transmit the pump data from the medical pump 1 to the smartphone 3 by means of the chip 2.

In step c, the pump data read out in step b is transmitted from the smartphone 3 to the service facility 4.

It should be understood that in step b, the transmission of pump data from chip 2 to smartphone 3 is independent of an existing Internet connection of pump 1. Of course, an existing data connection, in particular an Internet connection, is advantageous for transmitting the pump data from the smartphone 3 to the service facility 4.

In this example, pump 1 is configured to store pump data on chip 2 on a user-based and/or event-based basis. Chip 2 is protected against unauthorized reading of the pump data by smartphone 3 by at least one security feature, such as a security code.

In step c), which is shown in the Figure as a schematic dashed line c, the pump data is transmitted to the service facility 4. This data transmission is preferably encrypted. The pump data transmitted in step c is then evaluated in the service facility 4.

### REFERENCE CHARACTER LIST

1- Medical Pump

2- Component for contactless data transmission

21- NFC chip/RFID chip

3- Mobile terminal device

4- Service facility

1. A medical pump comprising a component for contactless data transmission of pump data to a mobile terminal device, wherein the component comprises the pump data.

2. The medical pump according to claim 1, wherein the component comprises an NFC chip and/or an RFID chip that is set up to read out the pump data via the mobile terminal device.

3. The medical pump according to claim 1, wherein the medical pump is configured to record the pump data on a user-based and/or event-based basis.

4. The medical pump according to claim 1, wherein the medical pump is configured to store the pump data on the component on a user-based and/or event-based basis.

5. The medical pump according to claim 1, wherein the pump data is selected from the group consisting of error data, error analysis data, device status data, device type data, performance data, time stamps, input logs, alarms, and information about a disposable item used.

6. The medical pump according to claim 1, wherein the component is protected by at least one security feature against unauthorized reading of the pump data via the mobile terminal device.

7. The medical pump according to claim 1, wherein the component is arranged in an area of the medical pump that is shielded from interference.

8. The medical pump according to claim 1, wherein transmission of pump data from the component to a mobile terminal device is independent of an Internet connection of the medical pump.

9. A method for wireless transmission of pump data, the method comprising the steps of:
providing the medical pump according to claim 1; and
transmitting the pump data from the medical pump to a mobile terminal device via the component.

10. The method according to claim 9, wherein the pump data is structured in a data packet suitable for transmission or storage.

11. The method according to claim 10, wherein the data packet is selected to represent a time segment around an event.

12. The method according to claim 10, wherein the data packet is selected to represent measured values relating to the medical pump.

13. The method according to claim 12, wherein the measured values relate to a drive or electronics.

14. The method according to claim 9, further comprising the step of transmitting the pump data from the mobile terminal device to a service facility.

15. The method according to claim 14, wherein the step of transmitting the pump data from the mobile terminal device to the service facility comprises encrypting a transmission of the pump data.

16. A mobile terminal device comprising means for executing an application program that, when executed, performs at least one of:
displaying pump data transmitted from a component for contactless data transmission from a medical pump to the mobile terminal device;
analyzing pump data transmitted from the component for contactless data transmission from the medical pump to the mobile terminal device; and
transmitting pump data transmitted from the component for contactless data transmission from the medical pump to the mobile terminal device to a service facility.

17. The mobile terminal device according to claim 16, wherein the application program, when executed, configures the mobile terminal device to filter and/or sort read data on the mobile terminal device.

18. The mobile terminal device according to claim 16, wherein the application program, when executed, configures the mobile terminal device to send a notification to a user once the data has been successfully transmitted and/or to add and transmit a user-defined comment on the data.

19. The mobile terminal device according to claim 16, further comprising an interface for integration with other hospital information systems.

20. A system for wireless transmission of pump data, the system comprising:
the mobile terminal device according to claim 16;
a medical pump comprising a component for contactless data transmission of pump data to the mobile terminal device, wherein the component comprises the pump data; and
a service facility that receives pump data from the mobile terminal device,
wherein the mobile terminal device serves as a reader for the pump data.
